(19) United States
(12) Patent Application Publication
(10) Pub. No.: US 2024/0315274 A1
(43) Pub. Date: Sep. 26, 2024

(54) AUTOMATED CLEAN IN PLACE SYSTEM FOR SOFT SERVE MACHINE

(71) Applicant: H.C. Duke & Son, LLC, East Moline, IL (US)

(72) Inventors: Dustin Meyermann, East Moline, IL (US); Kyle B. Elsom, Le Claire, IA (US); Michael A. Graef, Naperville, IL (US); Ji Won Elizabeth Oh, Chicago, IL (US); Brian Mathews, Sycamore, IL (US); Gary Paul Almblade, East Moline, IL (US)

(73) Assignee: H.C. Duke & Son, LLC, East Moline, IL (US)

(21) Appl. No.: 18/736,850

(22) Filed: Jun. 7, 2024

Related U.S. Application Data
(63) Continuation-in-part of application No. 17/068,266, filed on Oct. 12, 2020, now Pat. No. 12,029,224.
(60) Provisional application No. 62/913,763, filed on Oct. 11, 2019.

(51) Int. Cl. A23G 9/30; A23G 9/22; A23G 9/28; A61L 2/04; A61L 2/24; A61L 2/26; B08B 9/032

(52) U.S. Cl. CPC A23G 9/30; A23G 9/228; A23G 9/28; A61L 2/04; A61L 2/24; A61L 2/26; B08B 9/0321; A61L 2202/14; A61L 2202/17; B08B 2209/032

(57) ABSTRACT

A clean in place assembly for a soft serve ice cream dispenser includes an ice cream dispenser. A clean in place system having a water tank including a heater disposed therein is attached to the ice cream dispenser. A control interface is disposed on the tank. The control interface is connected to operating circuitry of the ice cream dispenser. A nozzle of the ice cream dispenser is formed of a conductive metal material and includes a notch formed therein circumferentially about a portion of the nozzle and includes radiused surfaces that extend to portions of the nozzle that house O-rings.

### CROSS-REFERENCE TO RELATED APPLICATIONS

This application is a continuation-in-part of U.S. patent application Ser. No. 17/068,266 filed Oct. 12, 2020, which in turn claims priority of U.S. Provisional Application No. 62/913,763 filed Oct. 11, 2019 the contents of both which are incorporated herein by reference in their entirety.

### FIELD OF THE INVENTION

The present disclosure relates to a process and apparatus for cleaning a soft serve machine.

### BACKGROUND OF THE INVENTION

Soft serve ice cream machines require cleaning, in some cases daily, in order to prevent bacteria growth. These cleaning cycles and their frequencies are mandated by local health codes. The cleaning procedures are developed by the manufacturer and certified by NSF to achieve the required reduction in bacterial count. Execution of the cleaning cycles requires the mechanical breakdown of the food contact areas of the machine to complete the sanitation. This includes the tear down and manual cleaning of the mix delivery system, the freeze cylinders, beater bars and dispense head. Cleaning cycles can occupy one to two employees for up to 1-2 hours. In addition, training and strict adherence to the established cleaning procedures is required to insure the effectiveness of the cleaning cycle. Employee turnover can present challenges to insure an adequate levels of trained employees to conduct the cleaning cycles.

There is therefore a need in the art for an improved cleaning procedure and apparatus to reduce the applied labor, and to improve the consistency and effectiveness of a cleaning cycle. There is a need for a unit to be cleaned without a mechanical breakdown of the machine, resulting in significant reductions in labor and improved consistency of the clean cycle.

### SUMMARY OF THE INVENTION

In one aspect, there is disclosed a clean in place assembly for a soft serve ice cream dispenser that includes an ice cream dispenser. A clean in place system having a water tank including a heater disposed therein is attached to the ice cream dispenser. A control interface is disposed on the tank. The control interface is connected to operating circuitry of the ice cream dispenser.

In another aspect, there is disclosed a method of sanitizing an ice cream dispenser comprising the steps of: providing an ice cream dispenser, providing a clean in place system, the clean in place system including a water tank including a heater disposed therein and a control interface disposed on the tank, the control interface connected to operating circuitry of the ice cream dispenser; performing a set up procedure; performing a rinse cycle preparation cycle; performing a rinse cycle; performing a heat preparation cycle wherein water in the tank is recycled in the tank and the heater is activated heating the water in the tank to a temperature of from 160 to 185 degrees Fahrenheit; and performing a heat cycle wherein the heated water is cycled to the ice cream dispenser for a predetermined amount of time sanitizing the ice cream dispenser.

In a further aspect, there is disclosed a clean in place assembly for a soft serve ice cream dispenser that includes an ice cream dispenser. A clean in place system having a water tank including a heater disposed therein is attached to the ice cream dispenser. A control interface is disposed on the tank. The control interface is connected to operating circuitry of the ice cream dispenser. A nozzle of the ice cream dispenser is formed of a conductive metal material and includes a notch formed therein circumferentially about a portion of the nozzle and includes radiused surfaces that extend to portions of the nozzle that house O-rings.

### BRIEF DESCRIPTION OF THE DRAWINGS

FIG. 1 is a graphical depiction of a clean in place system;

FIG. 2 is a graphical depiction of a clean in place system at initial setup;

FIG. 3 is a graphical depiction of a clean in place system at rinse cycle preparation;

FIG. 4 is a graphical depiction of a clean in place system at a rinse cycle;

FIG. 5 is a graphical depiction of a clean in place system at a heat cycle preparation;

FIG. 6 is a graphical depiction of a clean in place system at a heat cycle;

FIG. 7 is a graphical depiction of a clean in place system at a drain period;

FIG. 8 is a graphical depiction of a clean in place system at a final rinse and cooling;

FIG. 9 is a graphical depiction of a clean in place system at a dismount time;

FIG. 10 is perspective views of a dispense nozzle including geometry allowing flow in a clean cycle;

FIG. 11 is front and section views of the nozzle and an ice cream dispensing machine showing the nozzle in open and closed positions;

FIG. 12 is front and section views of the nozzle and an ice cream dispensing machine showing the nozzle in open and closed positions;

FIG. 13 schematically depicts a second aspect of the clean in place system of FIG. 1 according to one or more embodiments shown and described herein;

FIG. 14 schematically depicts an isolated front view of a tank of the clean in place system of FIG. 13 according to one or more embodiments shown and described herein; and

FIG. 15 schematically depicts an isolated exploded view of the tank of the clean in place system of FIG. 13 according to one or more embodiments shown and described herein.

### DETAILED DESCRIPTION OF THE PREFERRED EMBODIMENTS

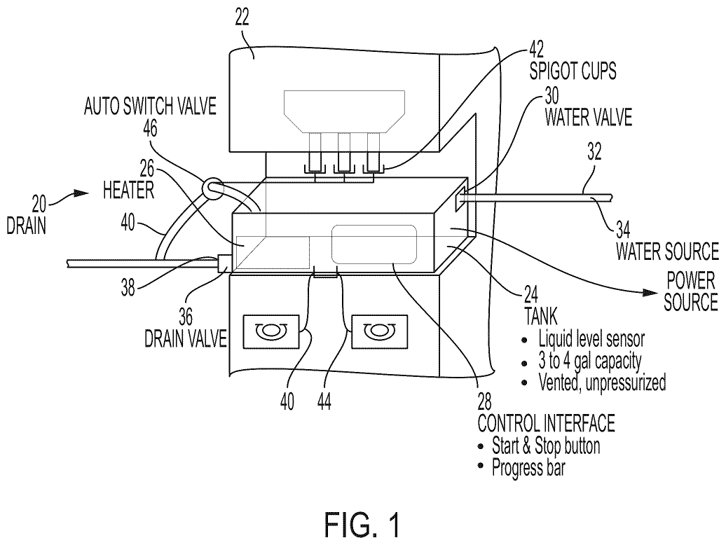

Referring to FIG. 1 there is shown a clean in place (CIP) system 20 for an ice cream dispenser 22. The CIP system 22 includes a tank 24 that has a heater 26 disposed therein. A control interface 28 is provided on the tank 24 to allow various functions of the system such as a start and stop as well as status of the system. The control interface 28 links to the operating circuitry of the ice cream dispenser allowing the CIP system 20 to control the various components of the ice cream dispenser 22 as will be discussed below.

The control interface 28 may include or be an electronic control unit (ECU), a central processing unit (CPU), and/or the like, for performing the functions as described herein. For example, the control interface 28 may be configured to receive, analyze and process sensor data, perform calculations and mathematical functions, convert data, generate data, and the like. The control interface 28 may include one or more processors and other components. For example, one or more memory modules that stores logic that is executable by the one or more processors. Each of the one or more processors may be a controller, an integrated circuit, a microchip, central processing unit or any other computing device. The one or more memory modules may be non-transitory computer readable medium and may be configured a RAM, ROM, flash memories, hard drives and/or any device capable of storing computer-executable instructions, such that the computer-executable instructions can be accessed by the one or more processors. The computer-executable instructions may include logic or algorithms, written in any programming language of any generation such as, for example machine language that may be directly executed by the processors, or assembly language, object orientated programming, scripting languages, microcode, and the like, that may be compiled or assembled into computer-executable instructions and storage on the one or more memory modules. Alternatively, the computer-executable instructions may be written in our hardware description language, such as logic implemented via either a field programmable gate array (FPGA) configuration or an application specific integrated circuit (ASIC), all their equivalents.

The tank 24 includes a water inlet 30 having a water valve 32 that is coupled to a water source 34. The tank 24 includes a drain valve 36 at an outlet 38 of the tank 24. Connections 40 are connected to the outlet 38 to mate with the spigots 42 of the ice cream dispensing machine as well as the mixing bag interface 44. A switch valve 46 may be positioned in the connections 40. The tank 24 includes a power connection 48 to a power source for the heater 26, as well as the control interface 28.

In one aspect, the control interface 28 integrates with the controls of the ice cream dispensing machine and specifically with the freezer controls. This integration enables the automation of the clean in place CIP cycle after minimal setup is performed, minimizing applied labor. Pump cycles, beater activations, are all controlled via the CIP controller interface 28 to the machine. This system integration also enables the implementation of machine lockouts such as the inability to dispense frozen product if clean cycles are not performed per required schedules. Clean cycle logs can also be generated.

The CIP system 20 utilizes hot water to improve the integrity of the clean cycle. Temperatures in excess of 150 F will effectively eliminate bacteria. This is optimal compared with trying to cover all surfaces with sanitizer. Sanitizer may also be used with the clean in place system, but more to augment than as a primary cleaning agent.

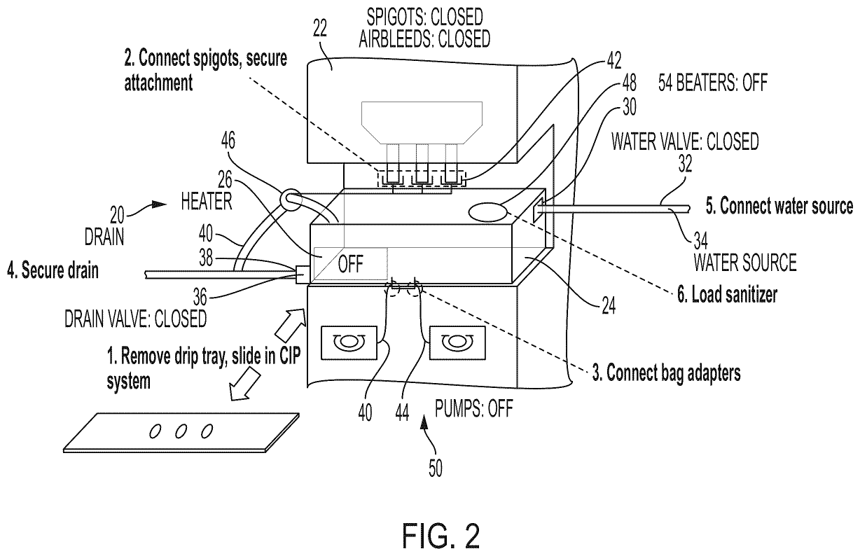

Referring to FIG. 2, the setup of the CIP system 20 is shown. The system is turned to a clean mode and ice cream product is dispensed from the barrels of the ice cream dispenser 22. The drip tray is removed from the ice cream dispenser 22 and the tank 24 is installed. The mix lines are disconnected from product bags. The connections 40 are attached to the spigot 42. The connections 40 are attached to the bag adapters 44 and to the outlet 38 of the tank 24. A water source 34 is connected to the inlet 30 of the tank 34 and sanitizer 48 is loaded into a compartment in the tank 24. A power source is coupled to the CIP system 20.

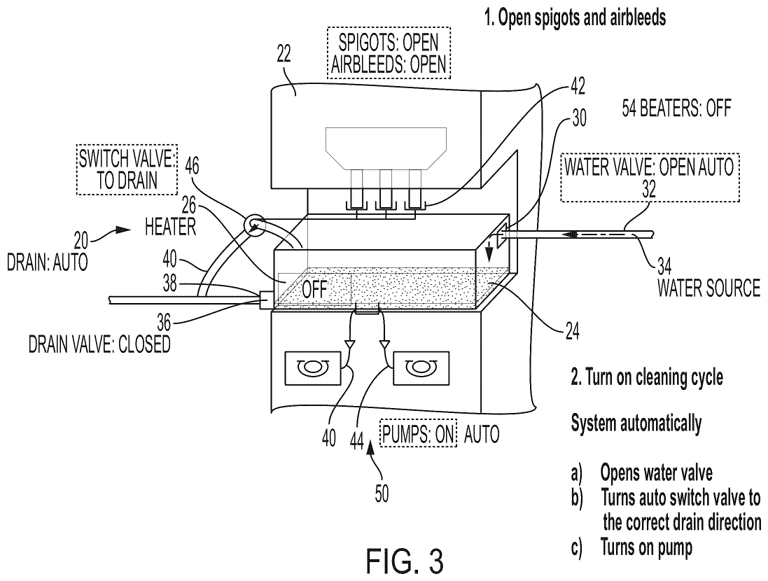

Referring to FIG. 3, the rinse cycle prep is shown. The spigots 42 and air bleed lines are opened. Water is allowed to enter the tank 24 through the inlet 30 into the tank 24 and a valve 36 at the outlet 38 of the tank 24 is closed. The pumps 50 associated with the ice cream dispenser 22 are turned on and water flows to the bag connections 44.

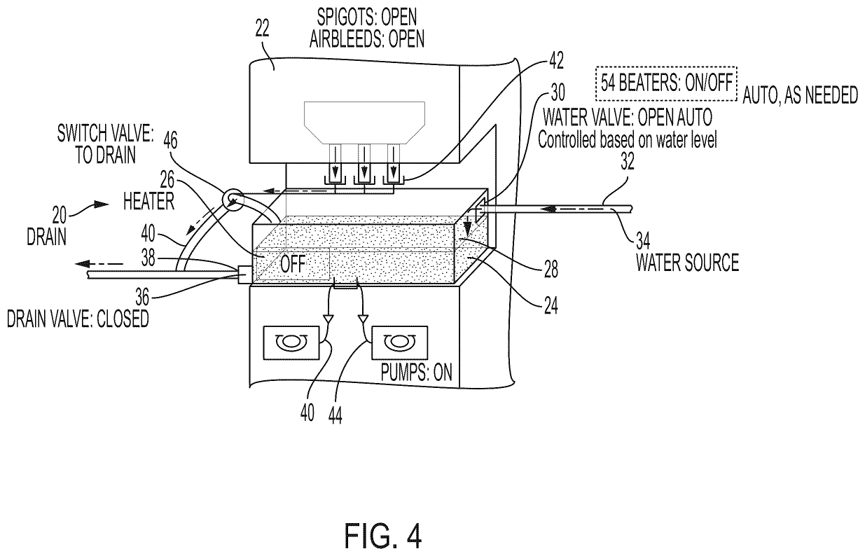

Referring to FIG. 4, the rinse cycle is shown. Water flows from the tank 24 through the bag connections 44 through the pumps 50 of the ice cream dispenser 22 to the cylinders and out through the spigots 42. The connections 40 at the spigots 42 direct the water to the switch valve 46 which routes the water to a drain 52. The beaters 54 of the ice cream dispenser 22 may be enabled or disabled as needed. The water runs through the system from 10 to 15 minutes until the water runs clear. In one aspect, the water may optionally be preheated by the heater to about 120 F and sanitizer may optionally be included in the water.

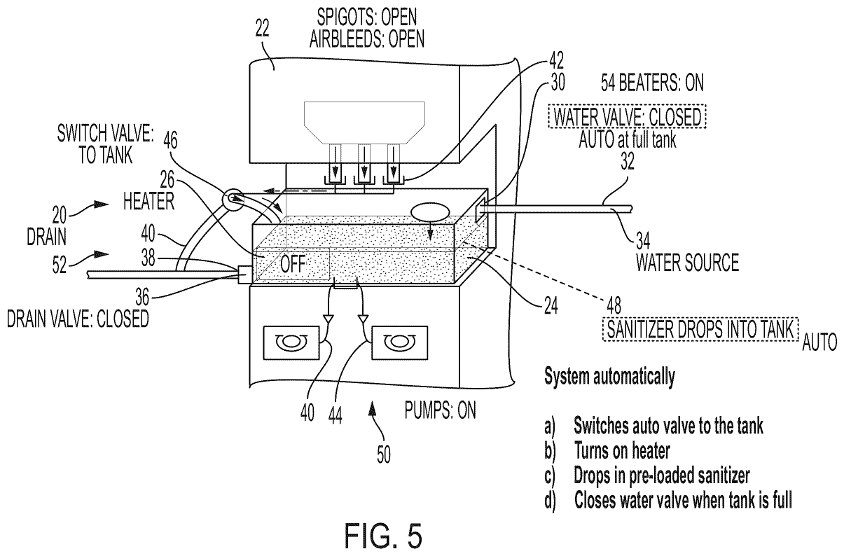

Referring to FIG. 5, the heat prep cycle is shown. The water valve at the tank inlet 30 is closed and the water level in the tank 24 is full. The heater 26 is turned on in the tank 24 and sanitizer may or may not be added 48 is added to the water in the tank 24. The switch valve 46 is moved to cycle water back to the tank 24 from the spigots 42.

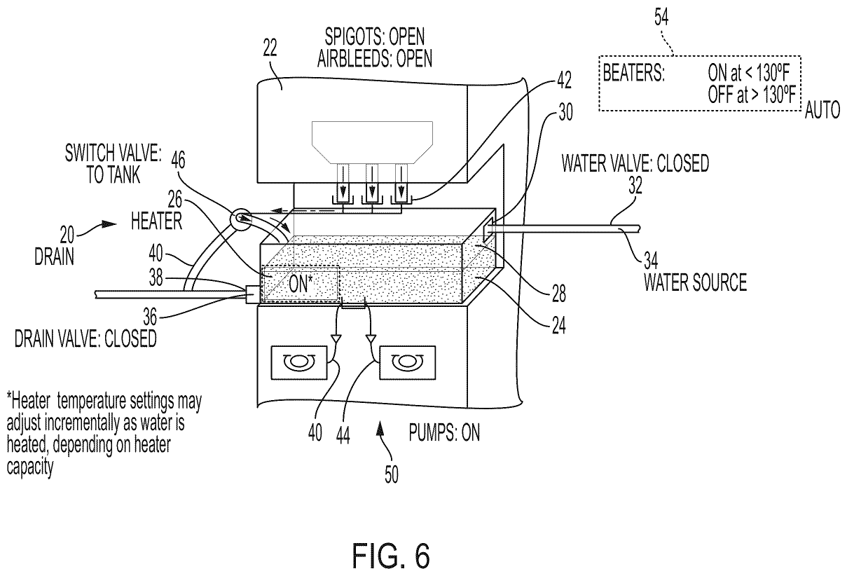

Referring to FIG. 6, the heat cycle is shown. Water is heated from 160 to 185 F in the tank 24 which forms a closed loop with the ice cream dispenser 22. The heated water flows through the ice cream dispenser 22 for a predetermined amount of time such as 40 minutes such that the surfaces of the ice cream dispenser 22 are heated to specified level. The beaters 54 of the ice cream dispenser 22 may be enabled or disabled as needed.

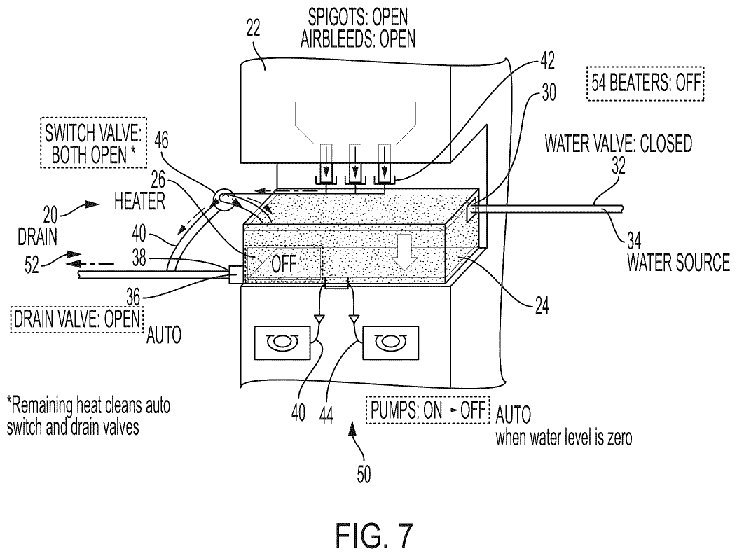

Referring to FIG. 7, the drain cycle is shown. The water valve at the inlet 30 of the tank 24 is closed. The heater 26 is deactivated and the switch valve 46 is actuated to the drain 52 and the tank 24. The drain valve to the tank is opened. The pumps 50 are run until all of the water has been removed from the tank 24.

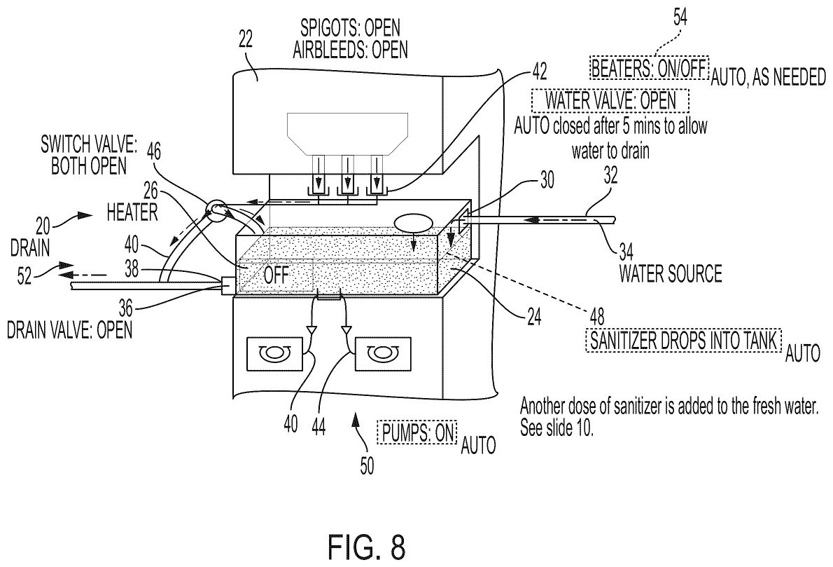

Referring to FIG. 8 an optional final rinse and cooling cycle is shown. The water valve at the inlet 30 of the tank 24 is opened and water enters the tank 24 for a specified amount of time until the tank is full. Sanitizer 48 may be added to the water. The switch valve 46 is actuated to the drain 52. The pumps 50 of the ice cream dispenser 22 are on and water flows through the ice cream dispenser 22 to cool the components to room temperature. The water may flow for a predetermined amount of time such as 10 minutes.

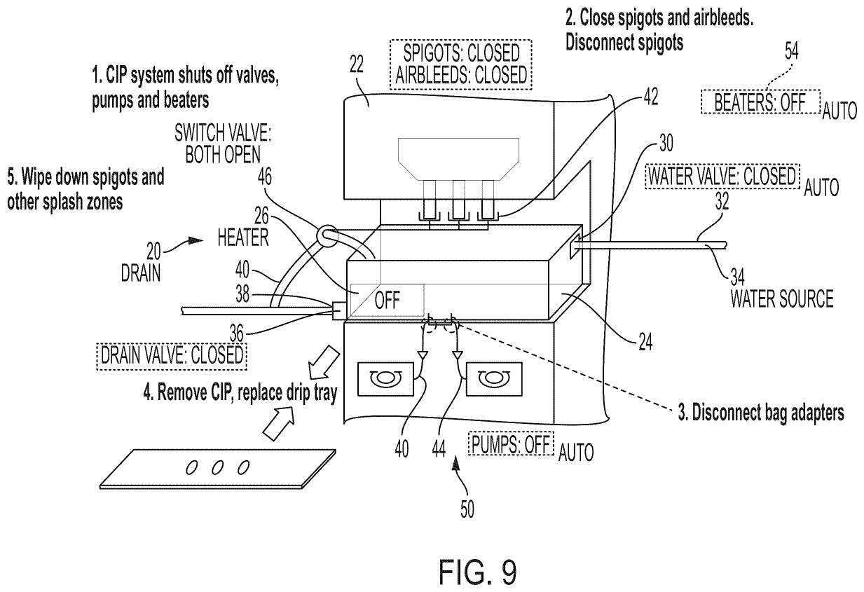

Referring to FIG. 9, the dismounting of the CIP system 20 is shown. The CIP system 20 shuts off the valves pumps and beaters. The spigots 42 and air bleeds are closed and the connections 40 are removed. The connections 40 at the bag adapters 44 are also removed. The tank 24 is then removed and the drip tray is reinstalled. Various splash zones may be wiped down.

The CIP system 20 will utilize power utilities from the ice cream dispenser 22 to support a self-contained heater module. This enables the CIP system to utilize tap water to supply the CIP system and generate hot water. This provides for an elevated rinse temperature cycle, 110-120 F, and for a sanitize cycle, 150-160 F water. The utilization of hot water in combination with the pump circuits from the freezer enables a frozen product cycle start. The prior art machines require the product to be liquefied before initiating a cleaning cycle. This can take up to 4 hours to accomplish, which constrains the store operator around cleaning operations. The use of hot water provides a superior sanitation cycle to that of chemical based sanitation. Once a uniform system temperature is reached for a pre-determined time, an effective sanitation cycle had been accomplished. This compares to chemical based sanitize cycles, which rely on water agitation and chemical contact of all surfaces to insure effective bacteria elimination.

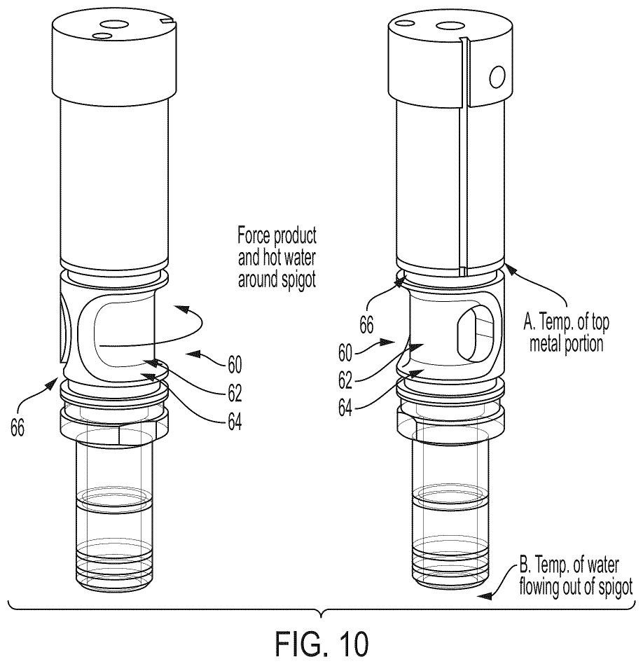

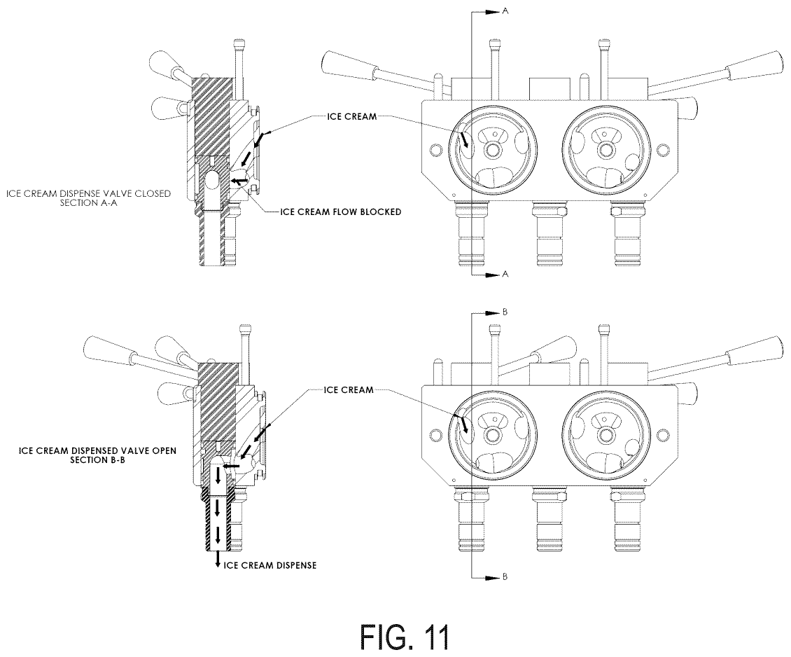

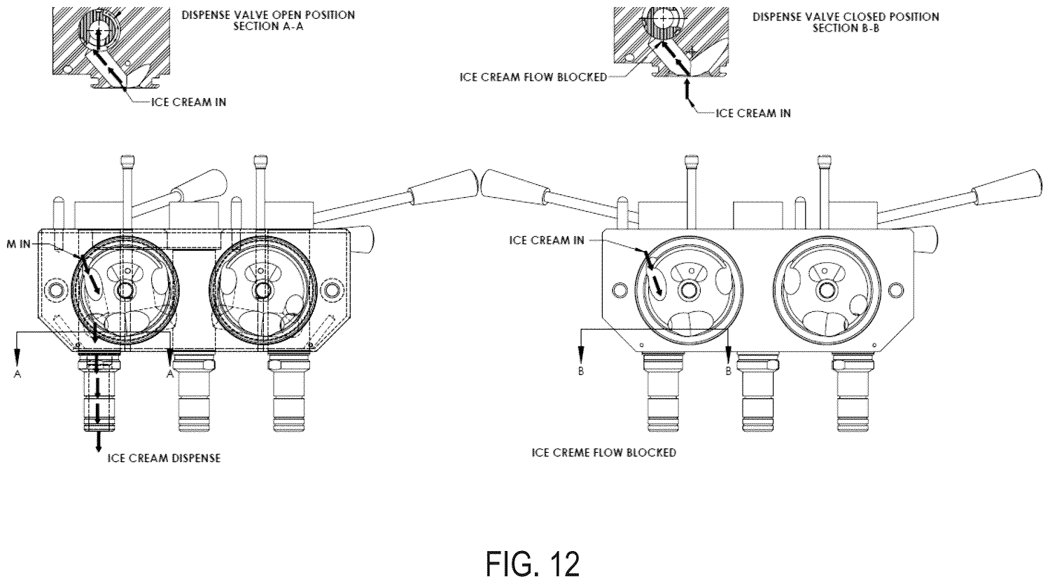

Referring to FIGS. 10-12, there is shown a nozzle 60 of an ice cream dispenser 22. The nozzle 60 is formed of a metal that can conduct heat. The nozzle 60 includes a notch 62 formed therein circumferentially about a portion of the nozzle 60. The notch 62 includes radiused surfaces 64 that extend to the portions of the nozzle that house O-rings 66. The notch 62 forces the frozen ice cream product or the cleaning water to flow around the spigot 42 allowing cleaning without multiple nozzle actuations. The nozzle 60 is actuated by rotation to allow flow or block flow as shown in FIGS. 11 and 12.

The ability to clean in place without removing or repeatedly activating plungers or nozzles is an advantage over the prior art. One of the more difficult areas to clean and sanitize are the dispense plungers. The plungers have O-rings, and are susceptible to mix getting trapped. These areas are difficult to clean. Prior art accomplished cleaning this area by rinsing and repeated activations of the plunger to purge any mix from trapped areas or requires removal of the plungers during the cleaning process and manually cleaning them. Both of these solutions add applied labor and manual processes which can affect the repeatability of a CIP process. To eliminate this, the nozzle having the notch described above enables the plunger to be cleaned with a single activation. The end user simply opens the plunger, and the CIP cycle is completed without having to remove or further activate.

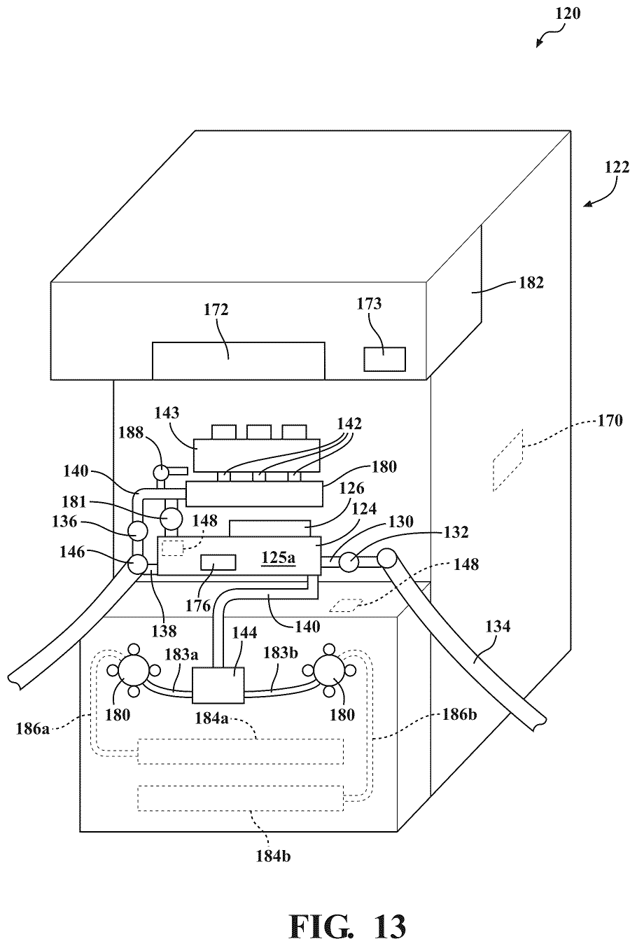

Now referring to FIGS. 13-15, a second aspect of a clean in place (CIP) system 120 for an ice cream dispenser 122 is schematically depicted. It is understood that the CIP system 120 is similar to the CIP system 20 described above with the exceptions of the features described herein. As such, like features will use the same reference numerals with a prefix “1” for the reference numbers. As such, for brevity reasons, these features will not be described again.

The CIP system 120 is integrated within the ice cream dispenser 122 to be a component of the ice cream dispenser 122. That is, the CIP system 120 is stationary or installed at the factory whereas the CIP system 20 is configured to be mobile or commonly removed from the ice cream dispenser 22. As such, the CIP system 120 advantageously does not need to be removed from the ice cream dispenser 122 after cleaning the ice cream dispenser 122, does not require users to relocated or space to store the tank and other components, and utilizes existing operating circuity of the ice cream dispenser 122, such as a control system 170 of the ice cream dispenser 122, to activate and display status of cycles of the CIP system 120.

In one embodiment, the CIP system 120 includes a clean in place control system 176 that is configured to be in communication with and utilize the existing operating circuity and electronic components of the ice cream dispenser 122 such as, without limitation, a freezer control 172 of the control system 170 of the ice cream dispenser 122, to activate and display status of cycles of the CIP system 120. That is, the clean in place control system 176 of the CIP system 120 utilizes the freezer control 172 of the control system 170 of the ice cream dispenser 122 itself, which further includes a user interface 173 to allow for the selection to activate the clean in place control system 176 of the CIP system 120 and to display status updates of the CIP system 120. In other embodiments, other aspects of the control system 170 of the ice cream dispenser 122 (e.g., different operating circuitry) are utilized to control the clean in place control system 176 of the CIP system 120.

Referring to FIG. 13, the CIP system 120 is schematically depicted. The CIP system 120 includes the tank 124 that has the heater 126 disposed therein. The clean in place control system 176 is attached or otherwise coupled to an exterior surface 125a of the tank 124. In one embodiment, the clean in place control system 176 is communicatively coupled to the freezer control 172 of the control system 170 of the ice cream dispenser 122. In other embodiments, the clean in place control system 176 is communicatively coupled to other aspects of the control system 170 of the ice cream dispenser 122 that is not the freezer control 172. As used herein, the term “communicatively coupled” means that coupled components are capable of exchanging data signals and/or electric signals with one another such as, for example, electrical signals via conductive medium, electromagnetic signals via air, optical signals via optical waveguides electrical energy via conductive medium or a non-conductive medium, data signals wirelessly and/or via conductive medium or a non-conductive medium and the like.

The clean in place control system 176 permits the CIP system 120 to control the various components of the ice cream dispenser 122 through the control system 170 and/or freezer control 172 of the ice cream dispenser 122 as will be discussed below.

The clean in place control system 176 may include or be an electronic control unit (ECU), a central processing unit (CPU), and/or the like, for performing the functions as described herein. For example, the clean in place control system 176 may be configured to receive, analyze and process sensor data, perform calculations and mathematical functions, convert data, generate data, and the like. The clean in place control system 176 may include one or more processors and other components. For example, one or more memory modules that stores logic that is executable by the one or more processors. Each of the one or more processors may be a controller, an integrated circuit, a microchip, central processing unit or any other computing device. The one or more memory modules may be non-transitory computer readable medium and may be configured a RAM, ROM, flash memories, hard drives and/or any device capable of storing computer-executable instructions, such that the computer-executable instructions can be accessed by the one or more processors. The computer-executable instructions may include logic or algorithms, written in any programming language of any generation such as, for example machine language that may be directly executed by the processors, or assembly language, object orientated programming, scripting languages, microcode, and the like, that may be compiled or assembled into computer-executable instructions and storage on the one or more memory modules. Alternatively, the computer-executable instructions may be written in our hardware description language, such as logic implemented via either a field programmable gate array (FPGA) configuration or an application specific integrated circuit (ASIC), all their equivalents. Accordingly, the methods and/or processes described herein may be implemented in any conventional computer programming language, as preprogrammed hardware elements, or as a combination of hardware and software components.

The tank 124 includes a water inlet 130 having a water valve 132 that is coupled to a water source 134. The tank 124 includes a drain valve 136 at an outlet 138 of the tank 124. A removable spigot manifold 180 is positioned to mate with the spigots 142 of the ice cream dispensing machine 122 positioned at a freezer dispense head 143 as well as a mixing bag interface 144. The mixing bag interface 144 may be fluidly coupled to a plurality of freezer pumps 180 of an ice cream freezer 182 via connections 183a, 183b, respectively. Further, each of the plurality of freezer pumps 180 may be fluidly coupled to a specific product bag 184a, 184, respectively, via mix lines 186a, 186b, respectively. The product bag 184a, 184b may hold or contain liquid product that is to be frozen within the ice cream freezer 182 passing through the corresponding freezer pump of the plurality of freezer pumps 180. The removable spigot manifold 180 includes connections 140 to fluidly connect the removable spigot manifold 180 to the outlet 138 such as at the switch valve 136.

That is, the switch valve 146 may be positioned in the connections 140. A recirculating valve 181 may be in fluid communication with the tank 124 and the connections 140 and may be configured to permit or inhibit the recirculation of water. The tank 124 further includes a power connection 148 that, in some embodiments, may receive power internally from the ice cream dispenser 122 to power the heater 126 and the clean in place control system 176. In other embodiments, the power connection 148 may be coupled to an external power supply to receive power, which in turn powers the heater 126 and the clean in place control system 176. As such, the power connection 148 may be any electronic component and may be configured to convert power between alternating current and direct current and vice versa.

In one aspect, the clean in place control system 176 integrates with the controls 170 of the ice cream dispensing machine 122 and may integrate with the freezer control 172. This integration enables the automation of the clean in place CIP cycle after minimal setup is performed, minimizing applied labor. Pump cycles, beater activations, are all controlled via the clean in place control system 176. This system integration also enables the implementation of machine lockouts such as the inability to dispense frozen product if clean cycles are not performed per required schedules. Clean cycle logs can also be generated.

In this aspect, when the CIP system 120 is turned to a clean mode, ice cream product is dispensed from the barrels of the ice cream dispenser 122. There is no need to remove the drip tray from the ice cream dispenser 122 nor is there a need to install the tank 124. The mix lines 186a, 186b, are disconnected from product bags 184a, 184b, respectively. The removable spigot manifold 180 is attached to the spigots 142 and connections 140 are optionally attached to the removable spigot manifold 180 (e.g., when there are not connections already on the removable spigot manifold 180) and to the tank 124. As such, during the rinse cycle, water flows out of the spigots 142 into and through the removable spigot manifold 180 to and through the connections 140. The connections 140 are attached to the mix lines 186a, 186b, and to the outlet 138 of the tank 124. A water source 134 is connected to the inlet 130 of the tank 134 and sanitizer 148 is loaded into a compartment in the tank 124.

With respect to deactivating the CIP system 120, the CIP system 120 shuts off the valves, pumps and beaters. The spigots 142 and an air bleed valve 188 are closed, the removable spigot manifold 180 and the connections 140 are removed from the bag adapter 144 and the mix lines 186a, 186b are disconnected from the bag adapter 144. There is no need to remove the tank 124 or install the drip tray in this aspect.

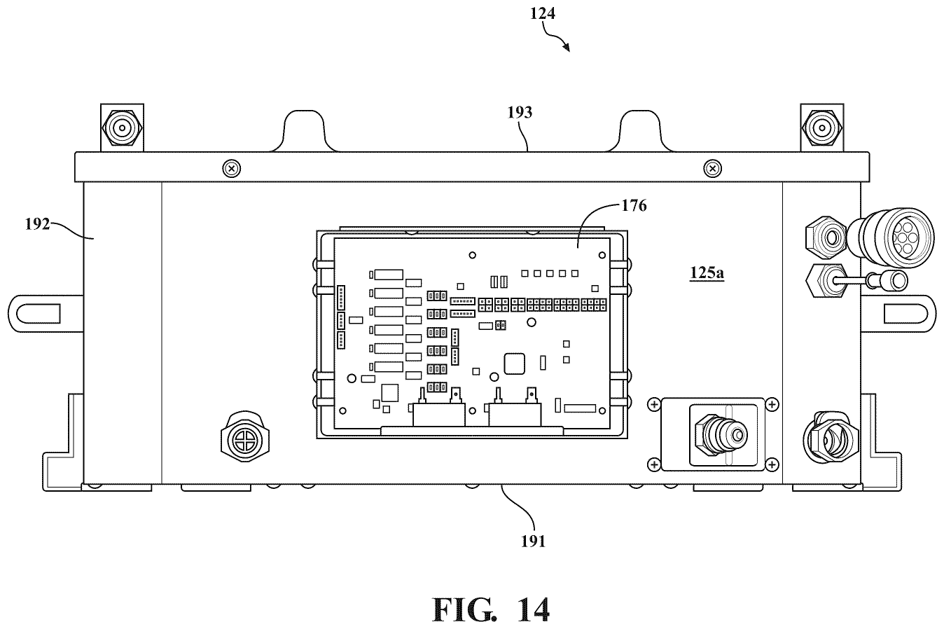

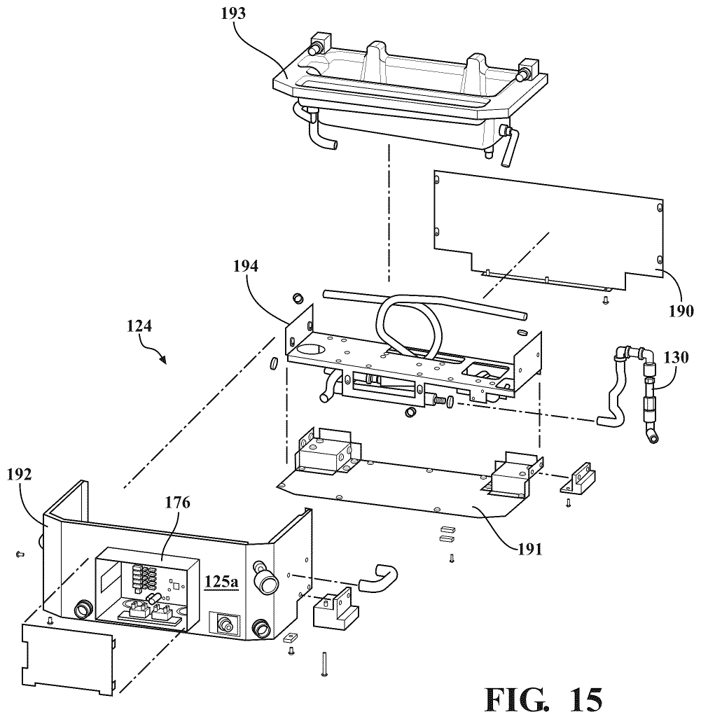

Now referring to FIGS. 14A-14B and 15, the tank 124 is defined by a rear panel 190, a lower panel 191 a front panel 192 and a tank assembly 193 that is a container for liquid, such as water and sanitizer. A plumbing assembly 194 is positioned within and between the rear panel 190, the lower panel 191, and the front panel 192.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A clean in place system for a soft serve ice cream dispenser assembly comprising:
an ice cream dispenser having a control system and a user interface; and
a clean in place assembly positioned within the ice cream dispenser, the clean in place assembly including:
a tank having a heater disposed therein; and
a clean in place control system communicatively coupled to the control system of the ice cream dispenser,
wherein the clean in place control system is activated via interfacing with the user interface of the ice cream dispenser.

2. The clean in place assembly for a soft serve ice cream dispenser assembly of claim 1 wherein the tank includes a water inlet having a water valve coupled to a water source and the tank including a drain valve at an outlet of the tank.

3. The clean in place assembly for a soft serve ice cream dispenser assembly of claim 2, further including at least one connection coupled to the outlet and the at least one connection coupled to spigots of the ice cream dispenser and coupled to a mixing bag interface of the ice cream dispenser.

4. The clean in place assembly for a soft serve ice cream dispenser assembly of claim 3, further including a switch valve positioned in the at least one connection.

5. The clean in place assembly for a soft serve ice cream dispenser assembly of claim 1, wherein the tank includes a power connection, the power connection coupled to a power source for the heater and the power connection coupled to the clean in place control system.

6. The clean in place assembly for a soft serve ice cream dispenser assembly of claim 1, wherein the clean in place control system is communicatively coupled to a freezer control of the ice cream dispenser.

7. The clean in place assembly for a soft serve ice cream dispenser assembly of claim 1, further including a lock out mechanism disposed in the ice cream dispenser, the lock out mechanism connected to the clean in place control system.

8. The clean in place assembly for a soft serve ice cream dispenser assembly of claim 1, further including a compartment formed in the tank, the compartment including sanitizer disposed therein.

9. The clean in place assembly for a soft serve ice cream dispenser assembly of claim 1, wherein a nozzle of the ice cream dispenser is formed of a conductive metal material and includes a notch formed therein circumferentially about a portion of the nozzle and including radiused surfaces that extend to portions of the nozzle that house O-rings.

10. The clean in place assembly for a soft serve ice cream dispenser assembly of claim 1, wherein the clean in place control system is positioned on an exterior surface of the tank.

11. The clean in place assembly for a soft serve ice cream dispenser assembly of claim 1, wherein the tank is integrated within the ice cream dispenser.

12. A method of sanitizing an ice cream dispenser assembly comprising the steps of:
providing an ice cream dispenser having a control system and a user interface;
providing a clean in place system, the clean in place system including a tank including a heater disposed therein and a clean in place control system communicatively coupled to the control system of the ice cream dispenser;
performing a set up procedure;
performing a rinse cycle preparation cycle;
performing a rinse cycle;
performing a heat preparation cycle wherein water in the tank is circulated in the tank and the heater is activated heating the water in the tank to a temperature of from 160 to 185 degrees Fahrenheit; and
performing a heat cycle wherein the heated water is cycled to the ice cream dispenser for a predetermined amount of time sanitizing the ice cream dispenser,
wherein the clean in place control system is activated via interfacing with the user interface of the ice cream dispenser.

13. The method of claim 12, wherein the set up procedure includes the steps of:
coupling the clean in place system to the ice cream dispenser;
disconnecting mix lines from product bags of the ice cream dispenser;
attaching connections to an outlet of the tank;
attaching connections to bag adapters of the ice cream dispenser;
attaching connections to a spigot of the ice cream dispenser;
connecting a water source to an inlet of the tank; and
connecting a power source to the clean in place system.

14. The method of claim 12, wherein the set up procedure includes the step of loading sanitizer into a compartment of the tank.

15. The method of claim 12, wherein the rinse cycle preparation cycle includes the steps of:
opening spigots and air bleed lines of the ice cream dispenser;
feeding water to the tank through an inlet into the tank;
closing a valve at an outlet of the tank; and
turning on pumps associated with the ice cream dispenser flowing water to bag connections of the ice cream dispenser.

16. The method of claim 12, wherein the rinse cycle includes the steps of:
flowing water from the tank through bag connections through pumps of the ice cream dispenser to cylinders and out through spigots;
directing connections at the spigots routing the water to a switch valve and to a drain; and
enabling and disabling beaters of the ice cream dispenser.

17. The method of claim 12, wherein the heat preparation cycle includes the steps of:
closing a water valve at an inlet of the tank;
activating the heater;
moving a switch valve cycling water to the tank from spigots of the ice cream dispenser.

18. The method of claim 12 wherein the heat preparation cycle further includes adding sanitizer to the water.

19. The method of claim 12, further including a drain cycle including the steps of:
closing a water valve at an inlet of the tank;
deactivating the heater;
actuating a switch valve to a drain and the tank;
opening a drain valve to the tank;
running pumps until all of the water has been removed from the tank.

20. A clean in place assembly for a soft serve ice cream dispenser assembly comprising:
an ice cream dispenser having a control system and a user interface, the ice cream dispenser having a nozzle of the ice cream dispenser is formed of a conductive metal material and includes a notch formed therein circumferentially about a portion of the nozzle and including radiused surfaces that extend to portions of the nozzle that house O-rings;
a clean in place assembly positioned within the ice cream dispenser, the clean in place assembly including:
a tank including a heater disposed therein;
a clean in place control system communicatively coupled to the control system of the ice cream dispenser,
wherein the clean in place control system is activated via interfacing with the user interface of the ice cream dispenser.
